(19) United States
(12) Patent Application Publication
(10) Pub. No.: US 2022/0079797 A1
(43) Pub. Date: Mar. 17, 2022

(54) APPAREL WITH INTEGRATED FEATURES FOR MUSCLE RECOVERY

(71) Applicant: Under Armour, Inc., Baltimore, MD (US)

(72) Inventor: Craig Santos, Portland, OR (US)

(21) Appl. No.: 17/019,171

(22) Filed: Sep. 11, 2020

(51) Int. Cl. A61F 5/03; A61F 5/02; A41D 13/005

(52) U.S. Cl. CPC A61F 5/03; A41D 13/0051; A61F 5/028

(57) ABSTRACT

An article of apparel may include a garment having a loose-fitting outer shell and an integrated compression band within the outer shell and configured to apply compression to a portion of the body of a wearer of the garment to promote muscle recovery. The compression band may be disposed internal to, and separate from, the outer shell of the garment.

### BACKGROUND

Compression is known to be used to aid in muscle recovery following vigorous exercise. For example, some athletes utilize compression sleeves to prevent swelling in their lower legs after vigorous athletic activity. Also, elastic garments may be made in small sizes to provide compression. For example, compression socks are often used by athletes after running or cycling to aid in recovery. However, compression sleeves and compression socks do not provide any aid in recovery for torso musculature, such as back muscles and abdominal muscles. In addition, compression sleeves and compression socks provide compression at all times. That is, with the sleeves or socks on, there is no way of turning the compression off. In addition, as separate articles, compression sleeves and compression socks must be put on separate and in addition to the wearer's conventional garments.

The present disclosure addresses one or more of the issues noted above.

### SUMMARY

In one aspect, the present disclosure is directed to an article of apparel. The article of apparel may include a garment having a loose-fitting outer shell and an integrated compression band within the outer shell and configured to apply compression to a portion of the body of a wearer of the garment to promote muscle recovery. The compression band may be disposed internal to, and separate from, the outer shell of the garment.

In another aspect, the present disclosure is directed to an article of apparel, including a garment having a loose-fitting outer shell and an integrated compression band within the outer shell and configured to apply compression to a portion of the body of a wearer of the garment. The compression band may include at least one shape memory alloy wire configured to selectively constrict in order to provide the compression to the portion of the body. In addition, the at least one shape memory alloy wire may be configured to apply heat to the body of the wearer.

In another aspect, the present disclosure is directed to an article of apparel, including a garment having a loose-fitting outer shell and an integrated compression band within the outer shell and configured to apply compression to a portion of the body of a wearer of the garment. The garment may be configured to be worn on the torso of the body. In addition, the compression band may include a fastening device disposed within a front pocket of the garment.

Other systems, methods, features, and advantages of the embodiments will be, or will become, apparent to one of ordinary skill in the art upon examination of the following figures and detailed description. It is intended that all such additional systems, methods, features, and advantages be included within this description and this summary, be within the scope of the embodiments, and be protected by the following claims.

### BRIEF DESCRIPTION OF THE DRAWINGS

The embodiments can be better understood with reference to the following drawings and description. The components in the figures are not necessarily to scale, with emphasis instead being placed upon illustrating the principles of the embodiments. Moreover, in the figures, like reference numerals designate corresponding parts throughout the different views.

FIG. 1 is a schematic front view of an article of apparel including a compression band according to an embodiment, with the compression band in a loosened condition;

FIG. 2 is a schematic rear view of the article of apparel of FIG. 1 with the compression band in a loosened condition;

FIG. 3 is a schematic front view of the article of apparel of FIG. 1 with the compression band in a tightened condition;

FIG. 4 is a schematic rear view of the article of apparel of FIG. 1 with the compression band in a tightened condition;

FIG. 5 is a schematic front view of an article of apparel with a compression band, showing a band control unit;

FIG. 6 is a schematic illustration of the compression band of FIG. 1 shown in isolation separated from the article of apparel;

FIG. 7 is a schematic front view of an article of apparel with a compression band, showing a cutaway portion of a front pocket revealing a fastening component of the compression band;

FIG. 8 is a schematic illustration of a muscle recovery system according to an embodiment;

FIG. 9 is a schematic illustration of a sweatsuit with limb compression bands according to an embodiment;

FIG. 10 is a schematic illustration of a compression band including a shape memory material wire and a separate heating element wire;

FIG. 11 is a schematic illustration of cutouts in an inner layer of a sweatshirt exposing a heating compression band;

FIG. 12 is a schematic illustration of a compression band being removed from a sweatshirt via the front pocket;

FIG. 13 is a schematic top-down view of an embodiment of a compression band to be incorporated into a garment; and

FIG. 14 is a schematic depiction of the compression band of FIG. 13 partially wrapped around the body of a wearer.

### DETAILED DESCRIPTION

The present disclosure is directed to articles of apparel. An article of apparel may include any of a variety of garments, such as shirts, sweatshirts, sweaters, jackets, pants, socks, undergarments, or any other article of clothing worn by a wearer.

For consistency and convenience, directional adjectives are employed throughout this detailed description corresponding to the illustrated embodiments. In addition, to assist and clarify the subsequent description of various embodiments, various terms are defined herein. Unless otherwise indicated, the following definitions apply throughout this specification (including the claims).

For purposes of this disclosure, the term “fixedly attached” shall refer to two components joined in a manner such that the components may not be readily separated (for example, without destroying one or both of the components). Exemplary modalities of fixed attachment may include joining with permanent adhesive, rivets, stitches, nails, staples, welding or other thermal joining, or other joining techniques. In addition, two components may be “fixedly attached” by virtue of being integrally formed, for example, in a molding process.

For purposes of this disclosure, the term “removably attached” shall refer to components that are joined in a manner such that the two components are secured together, but may be readily detached from one another. Examples of removable attachment mechanisms may include hook and loop fasteners, friction fit connections, interference fit connections, threaded connectors, cam-locking connectors, and other such readily detachable connectors. Similarly, “removably disposed” shall refer to the assembly of two components in a non-permanent fashion.

In accordance with the present disclosure, an article of apparel may include a garment having a loose-fitting outer shell and an integrated compression band within the outer shell and configured to apply compression to a portion of the body of a wearer of the garment to promote muscle recovery. In some embodiments, the compression band may be disposed internal to, and separate from, the outer shell of the garment.

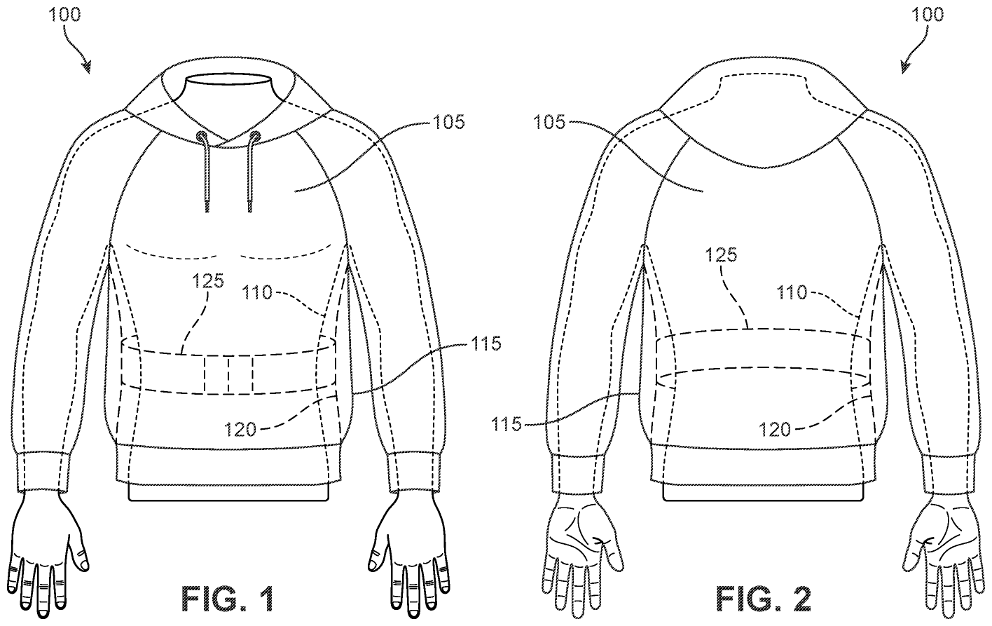

FIG. 1 is a schematic front view of an article of apparel including a compression band according to an embodiment, with the compression band in a loosened condition. As shown in FIG. 1, an article of apparel may include a garment 100. As shown in FIG. 1, in some embodiments, garment 100 may include a torso portion 105 configured to be worn on the torso of a wearer. For example, in some embodiments, garment 100 may be a sweatshirt, as shown in FIG. 1. In other embodiments, garment 100 may be a T-shirt, sweater, jacket, or other garment configured to be worn on the wearer's upper body 110. As shown in FIG. 1, garment 100 may have a loose-fitting outer shell 115. As illustrated in FIG. 1, outer shell 115 fits loosely on the wearer's body 110.

In addition, garment 100 may include an inner layer 120. As shown in FIG. 1, inner layer 120 may be disposed internal to, and separate from, outer shell 115, yet fixedly attached to outer shell 115 in select locations. Further, associated with inner layer 120 may be an integrated compression band 125 within outer shell 115. As shown in FIG. 1, compression band 125 may be disposed internal to, and separate from, outer shell 115 of garment 100. Compression band 125 may be configured to selectively apply compression to a portion of the body 110 of a wearer of garment 100 to promote muscle recovery. That is, as explained in further detail below, compression band 125 may be activated to constrict and, thereby, apply compression to the body of the wearer. In some embodiments, this functionality may be provided by shape memory alloy materials.

FIG. 2 is a schematic rear view of the article of apparel of FIG. 1 with the compression band in a loosened condition.

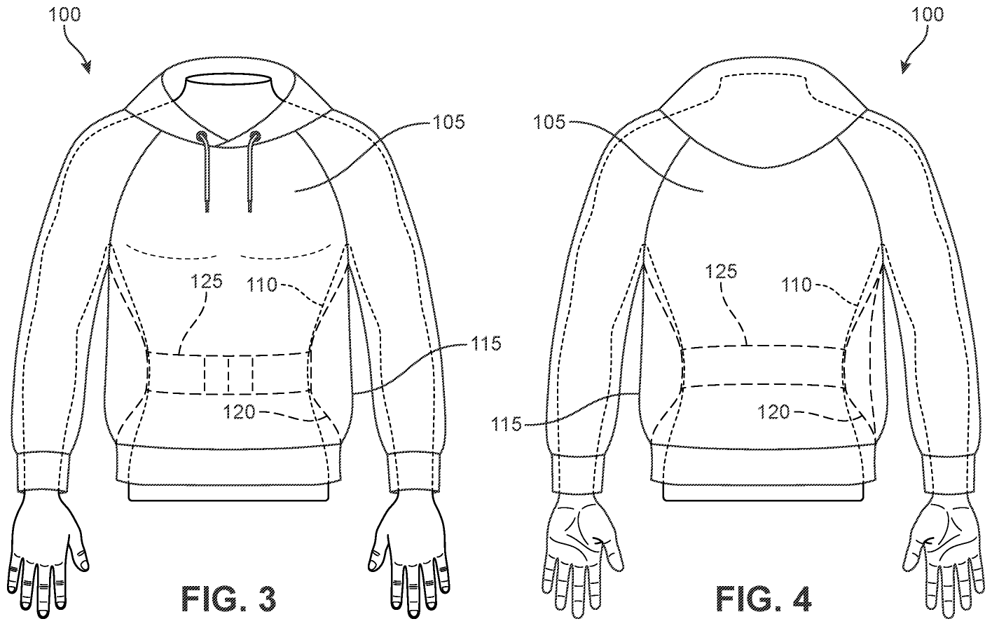

FIG. 3 is a schematic front view of the article of apparel of FIG. 1 with the compression band in a tightened condition. As shown in FIG. 3, compression band 125, upon activation, may constrict and provide compression and support to the torso. For example, as shown in FIG. 3, compression band 125 may be configured to apply compression to the lower back and abdominal region. In doing so, compression band 125 may promote recovery of lumbar back muscles and abdominal muscles. In addition, compression band 125 may also provide support to the wearer in the lumbar back and abdominal region.

FIG. 4 is a schematic rear view of the article of apparel of FIG. 1 with the compression band in a tightened condition.

In order to activate the compression band (e.g., by activating the shape memory alloy material), the garment may be provided with an integrated control unit. The control unit may include an activation switch. The control unit may be concealed from view, but accessible to the wearer. For example, in some embodiments, the control unit may be disposed in a pocket of the garment. This concealment of the activation switch maintains the regular appearance of the garment.

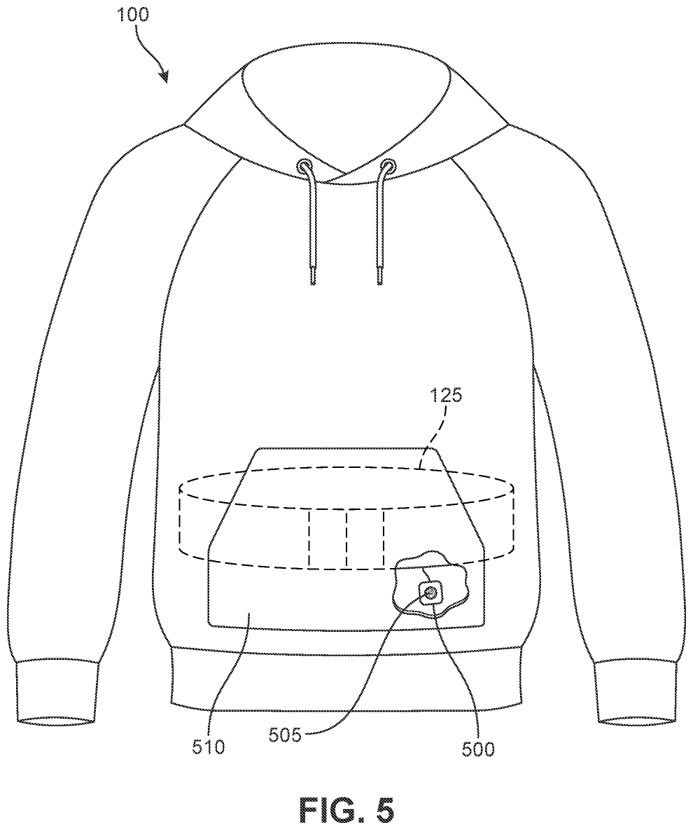

FIG. 5 is a schematic front view of an article of apparel with a compression band, showing a band control unit. As shown in FIG. 5, compression band 125 may be controlled by a control unit 500. Among other features, control unit 500 may include an activation switch 505 configured to activate compression band 125. As also shown in FIG. 5, in some embodiments, control unit 500 may be concealed, for example, within a pocket 510. It will be appreciated that, although control unit 500 is shown within a front pouch-type pocket, other types of pockets may be used to conceal control unit 500. Alternatively, in other embodiments, control unit 500 may be concealed and activation switch 505 may be exposed, for example, as a design element of garment 100. In still other embodiments, both control unit 500 and activation switch 505 may be exposed. By exposing one or both of control unit 500 and activation switch 505, activation may be facilitated. In addition, in some embodiments, these components may be made into aesthetic features of the garment.

In some embodiments, the compression band may include at least one shape memory alloy wire. The shape memory alloy wire may be activated, for example, by applying an electric current to the wire, which may shorten the length of the shape memory wire, thereby reducing the circumference of the hoop formed by the wire. By reducing the circumference of the wire hoop, the compression band may apply compression to a portion of the body.

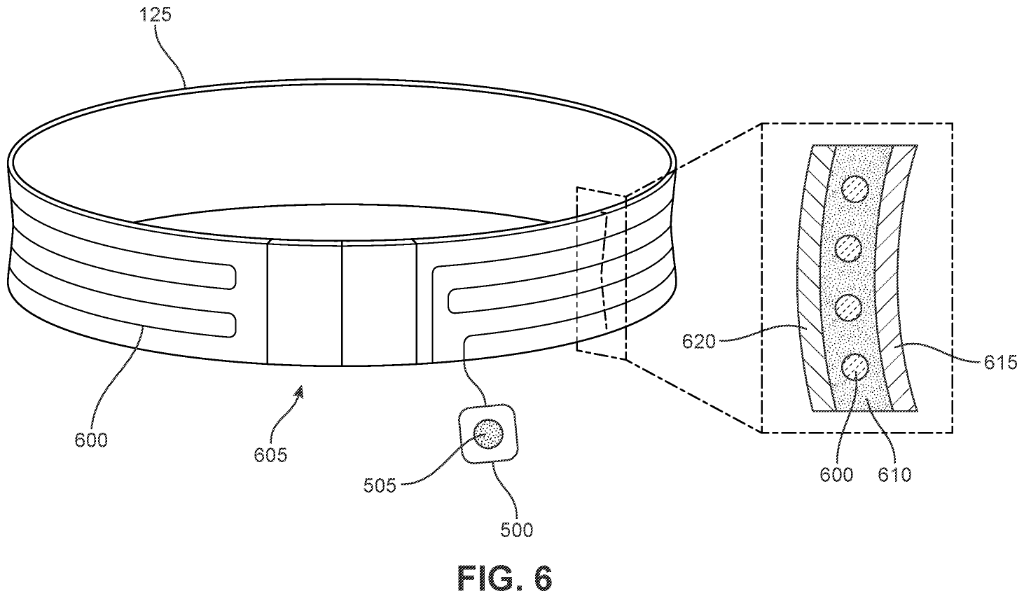

FIG. 6 is a schematic illustration of the compression band of FIG. 1 shown in isolation separated from the article of apparel. As shown in FIG. 6, compression band 125 may include at least one shape memory alloy wire 600 configured to selectively constrict in order to provide the compression to the portion of the body. For example, the shape memory alloy wire 600 may be configured to reduce in circumference upon application of an electric current. The activation of shape memory alloy wire 600 may be performed at a control device, such as control unit 500 with activation switch 505, which may be configured to receive input from a wearer of the article of apparel to activate the shape memory alloy wire 600.

In addition, in some embodiments, shape memory alloy wire 600 may be configured to apply heat to the body of the wearer. For example, in some embodiments, the shape memory alloy wire 600 may also serve as electrical heating elements. The application of heat to the body may provide therapeutic treatment, which may aid with muscle recovery. Also, in some embodiments, the application of heat may prepare an athlete for exercise and/or may be used during exercise to ensure that the wearer's muscles remain warm during activity.

As shown in FIG. 6, shape memory alloy wire 600 may be arranged in a serpentine configuration. This amplifies the amount of constriction provided when the length of shape memory alloy wire 600 is shortened upon the application of an electric current. This serpentine configuration also distributes the heat application more evenly across compression band 125.

FIG. 6 shows a cross-sectional view of compression band 125, illustrating multiple layers of compression band 125. For example, as shown in FIG. 6, compression band may include an middle layer 610 in which shape memory alloy wire 600 may be embedded. In addition, compression band may include an outer layer 615 and an inner layer 620.

Middle layer 610 may be formed of a material that facilitates the distribution of heat from shape memory alloy wire 600. For example, middle layer 610 may be formed of wool or a thermally conductive gel.

Outer layer 615 may be formed of an insulative material. For example, outer layer 615 may be formed of a thermally insulative plastic, gel, or fabric. In some embodiments, outer layer 615 may be formed of silicone, neoprene, or other suitable materials.

Inner layer 620 may be formed of a material that not only conducts heat but also provides comfort. For example, in some embodiments, inner layer 620 may be formed of fleece, cotton, or other comfortable fabric. In some embodiments, inner layer 620 may be formed by the inner layer of the garment in which it is disposed. In other embodiments, compression band 125 may be attached to the inner layer of the garment. Compression band 125 may be disposed on the exterior or interior of the inner layer of the garment.

It will be understood that the layers of compression band 125 may be flexible. The flexibility enables compression band 125 to conform to the contours of the body of the wearer. In addition the layers of compression band 125 may also be at least partially elastic. This elasticity facilitates the contraction and expansion of compression band 125 upon the activation and deactivation of shape memory alloy wire 600. It will also be understood that compression band 125 may have more or less layers than illustrated in FIG. 6. Additional or alternative layers may be included to provide comfort, elasticity, flexibility, padding, thermal distribution, thermal conduction, thermal insulation, or other properties.

In some embodiments, compression band 125 may be a continuous, uninterrupted loop. In other embodiments, as shown in FIG. 6, compression band 125 may include a fastening device 605, such as a buckle, latch, or other mechanism, to attach ends of compression band 125.

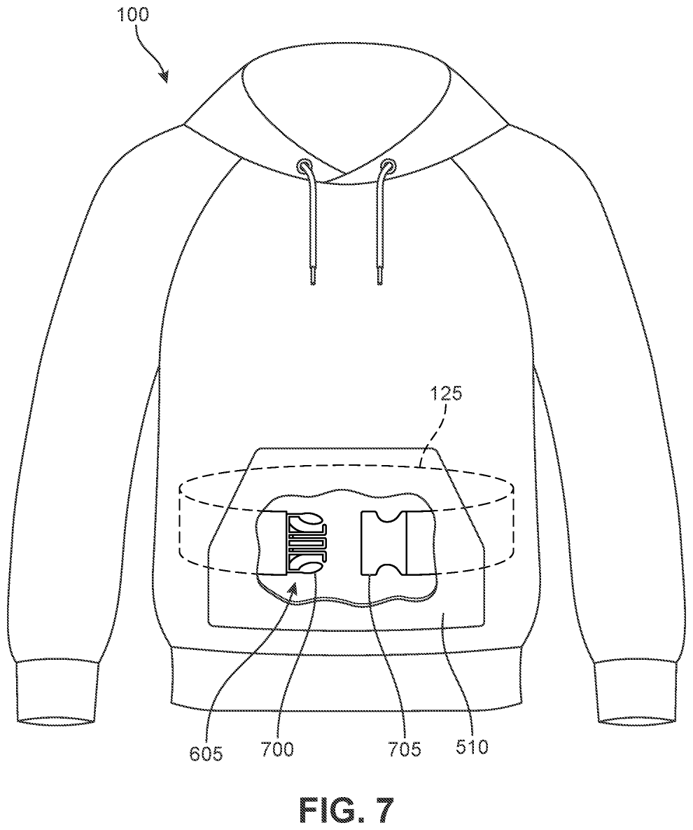

In some embodiments, fastening device 605 may be concealed, for example within a pocket of a garment. FIG. 7 is a schematic front view of an article of apparel with a compression band, showing a cutaway portion of a front pocket revealing a fastening device of the compression band. As shown in FIG. 7, in some embodiments, fastening device 605 may be concealed within pocket 510. Fastening device 605 may include a first component 700 attached to a first end of compression band 125 and a second component 705 attached to a second end of compression band 125. First component 700 may be fastenable to second component 705.

In some embodiments, an activatable compression band (e.g., with shape memory alloy wire) may include a fastening device 605. In other embodiments, fastening device 605 may be utilized with an elastic compression band, such that, when the components of fastening device 605 are secured to one another, the elastic compression band applies compression to the body part of the wearer around which the elastic compression band is disposed.

In some embodiments, the shape memory material may be configured to shift into the shape of a supportive element. For example, rather than simply constrict, the shape memory material may change shape into a supportive lumbar element, similar to a weight lifting belt. This shape-shifting capability may be utilized to provide various types of posture control/support in different areas of the body.

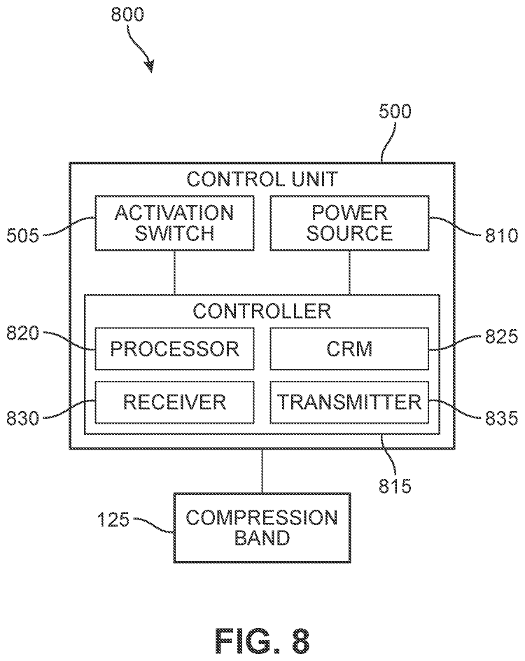

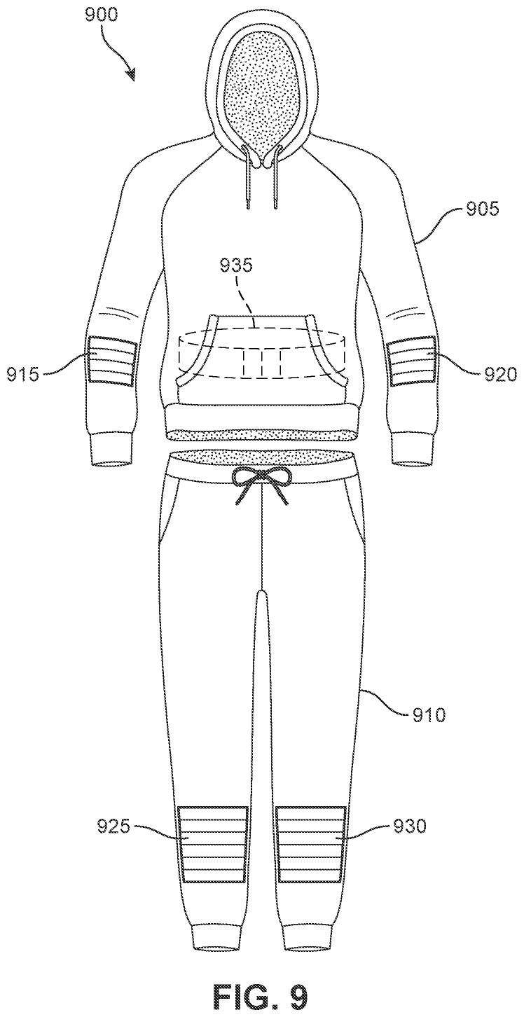

FIG. 8 is a schematic illustration of a muscle recovery system according to an embodiment. As shown in FIG. 8, a muscle recovery system 800 may include a compression band 125 and a control unit 500, both of which may be integrated with an article of apparel. Compression band 125 may have any of the features described above. As also described above, control unit 500 may include an activation switch 505. In addition to activation switch 505, control unit 500 may include a power source 810. Power source 810 may include, for example, one or more batteries. In addition, control unit 500 may include a controller 815 configured to control operation of control unit 500 in order to regulate constriction of compression band 125.

Controller 815 may include various computing and communications hardware. For example, as shown in FIG. 8, controller 815 may include a device processor 820 and a non-transitory computer readable medium 825 including instructions executable by device processor 820. Computer readable medium 825 may include any suitable computer readable medium including, for example, a memory, such as RAM, ROM, flash memory, or any other type of memory known in the art.

Further, controller 815 may include a receiver 830 and a transmitter 835. (It will be appreciated that, in some embodiments, the receiver and transmitter may be combined in a transceiver.) Receiver 830 and transmitter 835 may be configured to provide communication with other nodes of system 800. For example, receiver 830 may be configured to receive activation instructions from activation switch 505, and transmitter 835 may be configured to provide the instructions to power source 810 to supply the electric current to compression band 125.

In some embodiments, garments may be provided with compression bands elsewhere besides in areas that correspond with the torso of the wearer. For example, in some embodiments, compression bands may be provided in arm sleeves of a loose-fitting shirt, sweatshirt, jacket, etc. In some embodiments, compression bands may be provided in the pant legs of loose-fitting pants. Thus, the garment may include at least one tubular sleeve configured to receive a limb of a wearer. The compression band may be located within the tubular sleeve and may be configured to apply compression to the limb of the wearer.

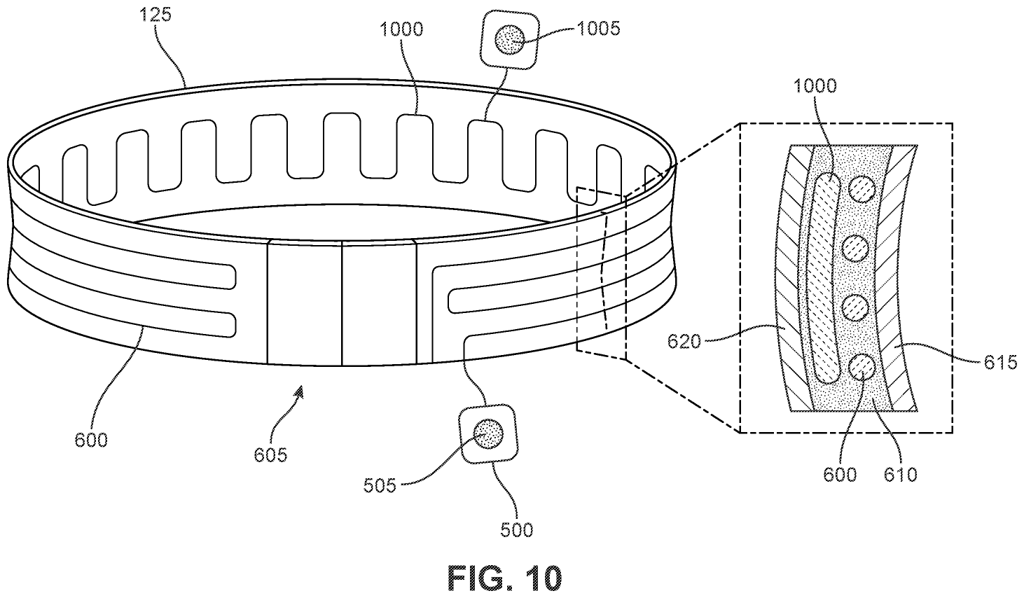

FIG. 9 is a schematic illustration of a sweatsuit with limb compression bands according to an embodiment. As shown in FIG. 9, an article of apparel may include garments, such as a sweatsuit 900. Sweatsuit 900 may include a sweatshirt 905. In some embodiments, sweatshirt 905 may be relatively loose-fitting, and may include integrated compression bands, such as a first compression band 915 integrated into a first sleeve and a second compression band 920 integrated into a second sleeve. These compression bands may be incorporated into inner layers of the sleeves, as described above with respect to the torso compression band in FIGS. 1-4. These inner layers may be internal to, and separate from, the external, loose-fitting shell of sweatshirt 900, yet fixedly attached to the shell in select locations.

It will be understood that sweatshirts may be provided with a compression band on only one sleeve. Such a single compression band sweatshirt may be preferred, for example, by a baseball pitcher, football quarterback, or tennis player, who may only need compression on one of their arms.

As also shown in FIG. 9, sweatsuit 900 may also include sweatpants 910. Sweatpants 910 may include a loose-fitting outer shell, and may include one or more compression bands integrated into the pant legs. For example, sweatpants 910 may include a first compression band 925 and a second compression band 930. These compression bands may have any of the features described with respect to other embodiments above. For example, these compression bands may be integrated with inner layers of the respective pant legs, such that the compression bands are internal to, and separate from, the loose-fitting outer shell of sweatpants 910.

It will be understood that sweatshirt 905 or sweatpants 910 could be provided separate from one another, and need not be included together as a complete sweatsuit. It will also be understood that, in some embodiments, only a single pant leg may include a compression band. In addition, it will be understood that, in both the sweatshirt and sweatpants, the compression bands may have any suitable length, i.e., to apply compression to as much or as little of the arms/legs as desired.

It is also noteworthy that these compression bands are generally disposed proximate the distal end of the wearer's limbs. This is to push fluids, like swelling and lactic acid, out of the limbs, in order to aid with muscle recovery.

As shown in FIG. 9, in some embodiments, apparel that includes compression bands for limbs may also include a torso compression band 935. Both the limb compression bands and torso compression band 935 may be configured as any of the compression bands disclosed herein.

The compression bands disclosed herein are concealed within the garments so that the garments appear to be regular articles of clothing. This provides wearers with the option to not reveal their use of compression bands, or simply to maintain a fashion sense not influenced by the bands. At the same time, including the compression bands integrated with the garments provides convenience. That is, the wearer need not put on compression bands as separate articles, nor need they carry them around prior to the games. In fact, the wearer may wear the same sweatshirt and pants before a game without the compression bands activated, and may simply change back into the same sweatshirt and pants after the game, at which point they may activate the compression bands to promote muscle recovery.

In some embodiments, compression bands may be provided with separate heating elements. That is, in some cases, the shape memory material that provides the constriction may be separate from the heating element. For example, a separate conductive wire, such as a copper wire, may be included. In such cases, the conductive wire that provides heating may be positioned on the side of the compression band that is closest to the body of the wearer. In some embodiments, the separate heating element may be controlled independently of the constricting shape memory material.

FIG. 10 is a schematic illustration of a compression band including a shape memory material wire and a separate heating element wire. As shown in FIG. 10, compression band 125 may include a heating element 1000 that is separate from shape memory alloy wire 600. As further shown in FIG. 10, heating element 1000 may be provided on the inner side of compression band 125, in order to better conduct heat to the body of the wearer.

As shown in FIG. 10, in some embodiments, heating element 1000 may have a sinusoidal or oscillating configuration. This sinusoidal configuration enables heating element 1000 to expand and contract as the shape memory alloy wire 600 constricts and relaxes compression band 125.

FIG. 10 also shows a separate heating element control device 1005, which may be configured to regulate the amount of current sent through heating element 1000, thus controlling the amount of heat generated by heating element 1000. It will be understood that heating element control device 1005 is depicted schematically and that the actual configuration and placement may be selected according to a skilled artisan. In some embodiments, heating element control device 1005 may be integrated with control unit 500 configured to control shape memory alloy wire 600.

In some embodiments, a band may be provided that includes a heating element without a constricting shape memory alloy wire. In some cases, such a band may be made of an elastic material to secure the band against the body in order to conduct heat to the body of the wearer.

In order to facilitate the conduction of heat from the compression band to the body of the wearer, one or more layers of the garment may include cutouts in order to expose the compression band to the body of the wearer. In some embodiments, the cutouts may be provided in the area in which the band is disposed. In some embodiments, an entire layer between the compression band and the body may be formed of a mesh type material.

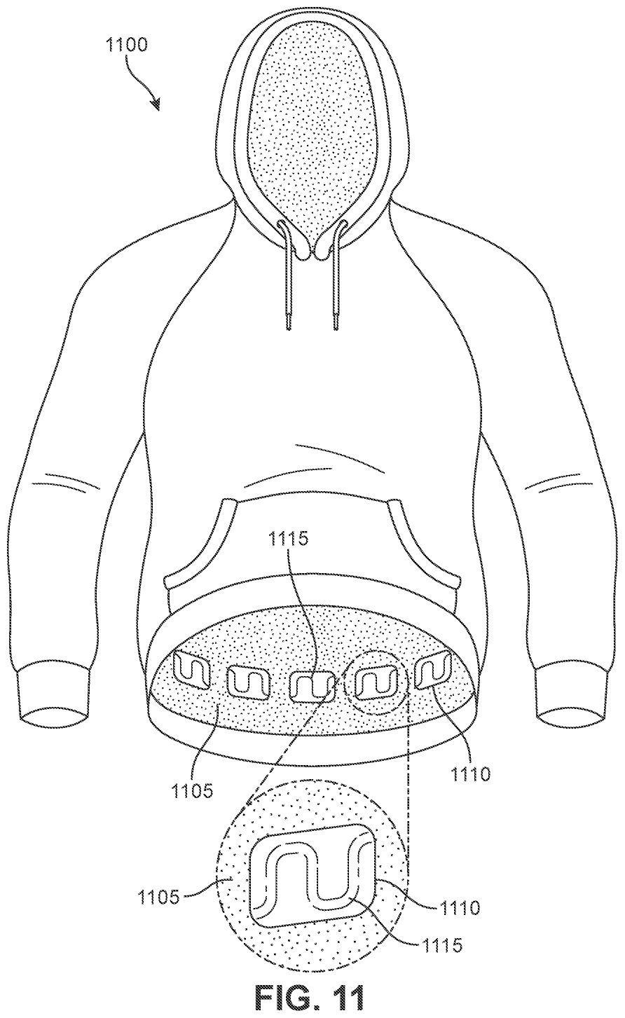

FIG. 11 is a schematic illustration of cutouts in an inner layer of a sweatshirt exposing a heating compression band. As shown in FIG. 111, a sweatshirt 1100 is depicted, revealing an inner layer 1105 of the garment. This inner layer is similar to layer 120 discussed above. That is, inner layer 1105 may be an inner layer within a loose-fitting outer shell. As shown in FIG. 11, inner layer 1105 may include one or more cutouts 1110 exposing a compression band to the body of the wearer. For example, as shown in FIG. 11, a heating element 1115 is shown through cutouts 1110. It will be understood that heating element 1115 may be embedded within the layers of the compression band itself (see e.g., the cross-sectional views of FIG. 6 and FIG. 10). Accordingly, as shown in the enlarged portion of FIG. 11, heating element 1115 is shown as a bulge or ridge created in a covering layer of the compression band.

In some embodiments, the compression band may be removable from the garment. This may facilitate washing of the garment and/or the compression band. For example, in some embodiments, the compression band may require hand washing (so as not to damage electronics) while the remainder of the garment may be machine washed.

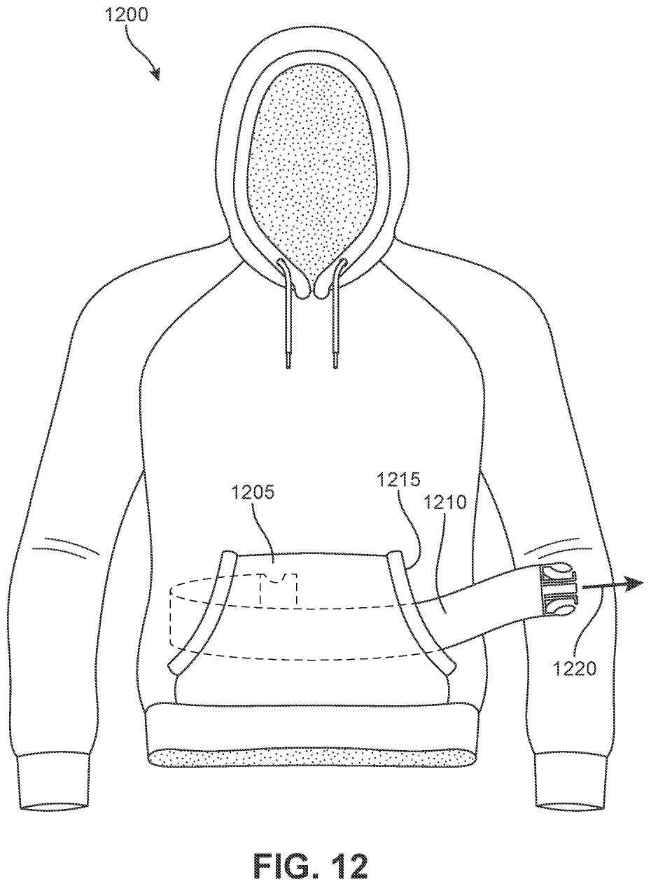

FIG. 12 is a schematic illustration of a compression band being removed from a sweatshirt via the front pocket. As shown in FIG. 12, a sweatshirt 1200 may include a front pocket 1205. A compression band 1210 may be incorporated into sweatshirt 1200, similar to that shown in FIG. 7. In the embodiment shown in FIG. 12, compression band 1210 may be removable from the garment, as shown by arrow 1220 as compression band 1210 is pulled from an opening 1215 of pocket 1205. In order to facilitate this removability, compression band 1210 may be disposed within a sleeve or tube incorporated into the inner layer of the sweatshirt. That is, compression band may not be stitched or adhesively attached to the inner layer of the sweatshirt.

Some embodiments may incorporate concepts from Santos, U.S. patent application Ser. No. 16/880,628, filed May 21, 2020, and entitled “Wrap Compression System,” the entire disclosure of which is incorporated herein by reference. For example, in some embodiments, the compression band may have an overlapping or double-wrapped configuration. This double-wrapping multiplies the constriction provided by the shape memory wire. This configuration also reduces the amount of electrical energy required to provide the desired constriction.

As a general matter, a wrap can utilize shape changing materials to apply compression. It may also contain material that, separate from or the same as the shape changing material, conducts and releases heat. For example, the shape changing materials can include a shape memory metal alloy implemented as a shape memory wire (e.g., Nitinol wire). The shape changing elements (“wiring”) can be operable to change shape in response to an external stimulus. This change of shape effectively reduces the circumference of the wrap encircling the user, thereby applying pressure or a compressive force to the user. In some embodiments, the wiring is an element configured to change length, and more particularly to reduce its length in response to the stimulus. The wiring can be one or more wires formed of a “shape memory” material or alloy that shrinks when a current is applied to the wire, and that returns to its original “memory” configuration when the current is removed or changed. In some embodiments, the wiring can include a wire formed of a “memory” material that changes length upon application of an electrical signal and then returns to its original length when the signal is terminated. Thus, the wiring activated and deactivated to create varying amounts of compressive forces on a user. The memory material can be a memory metal such as Nitinol. In other embodiments, electroactive polymers (EAP) can also be used in place of the Nitinol wires. EAPs are polymers that can exhibit a change in size and/or shape when stimulated by an electric field.

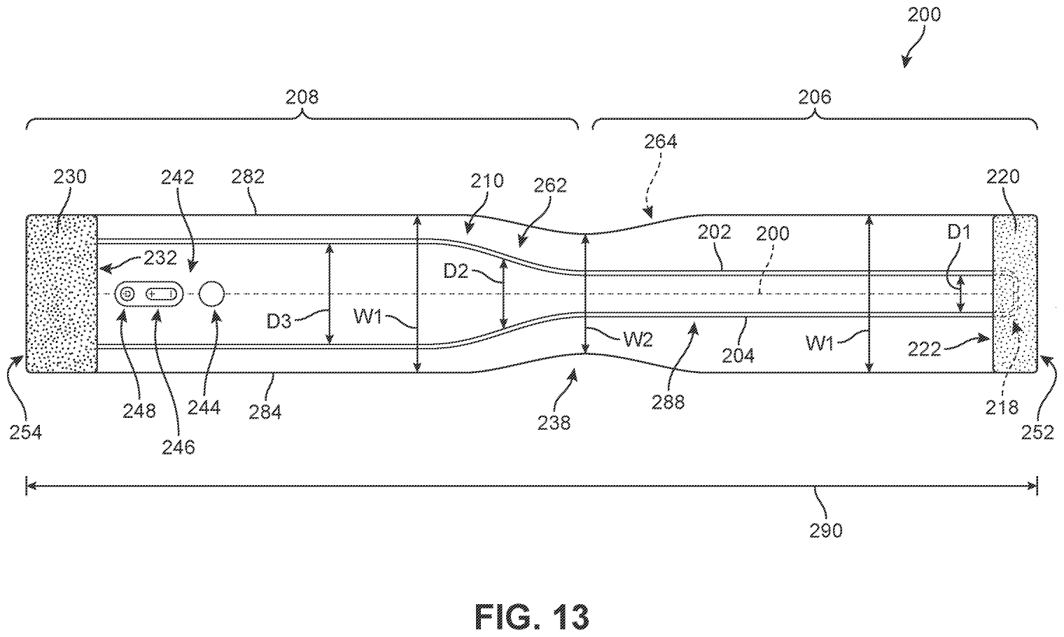

FIG. 13 is a schematic top-down view of an embodiment of a compression band to be incorporated into a garment. Such a compression band may be incorporated into an article of apparel as discussed with respect to other embodiments above. For purposes of clarity, FIG. 13 presents a top-down view of an outwardly-facing surface side (“outward side”) 262 of a compressive appliance 200. For reference, opposite inwardly-facing surface side (“inward side”) 264 is designed to face toward the user's skin, while outward side 262 is designed to face away from the user's skin. As shown in FIG. 2, compressive appliance 200 can include a plurality of components, including an elongated wrap portion (“wrap”) 210, a control interface 242, two end tab portions (a first end portion 220 and a second end portion 230), and wiring 288 embedded within the wrap 210 and the end portions.

While the compressive appliance 200 may be understood to extend fully across with a length 290 between a first end 252 and a second end 254, the wrap 210 itself only extends longitudinally between a first edge 222 and a second edge 232, where the first edge 222 borders the first end portion 220 and the second edge 232 borders the second end portion 230. The wrap 210 also extends in a lateral direction between an upper edge 282 and a lower edge 284. In addition, one or both end portions 220 and 230 can include one or more fastening mechanisms configured to secure the compressive appliance 200 once it has been wrapped around a body part. It will be understood that, in some embodiments, compressive appliance 200 may be fixedly attached to the garment in the double-wrapped configuration.

The wrap 210 further includes a wiring arrangement in which a single wire extends from the electronic assembly housed primarily within second end portion 230. The wiring extends across the length of the wrap 210 and loops back within the first end portion 220 itself (e.g., see a loop terminus 280 indicated by dotted line), or elsewhere within the wrap 210, such that the wiring 288 can be understood to be doubled, forming two distinct pathways across the length of the wrap 210. As noted earlier, because the nitinol wires feature a limited coefficient of contraction, the repeated looping multiplies the compressive force and offers compression benefits similar to or greater than those experienced by wraps in which additional wiring lengths are integrated into the length of the wrap.

As shown in FIG. 13, an upper wire segment 202 of the wiring 288 runs along the upper region (i.e., relative to a midline 298) of the wrap 210, and a lower wire segment 204 of the wiring 288 runs along the lower region (i.e., relative to the midline 298) of the wrap 210. While the two segments are identified separately for purposes of reference, it should be understood that together they comprise a single (continuous) wire element. In some embodiments, the upper wire segment 202 and lower wire segment 204 are arrange symmetrically about the midline 298 (i.e., mirror-image). In other words, as a general matter, the spacing between the upper wire segment 202 and the midline 298 is substantially equal to the spacing between the lower wire segment 204 and the midline 298, allowing for a more even compressive distribution across the wrap.

In addition, to better appreciate some of the benefits provided by the compressive appliance 200, the wrap 210 has been demarcated into two zones, including a first zone 206 and a second zone 208, where the first zone 206 refers to the portion of the wrap 210 designed to initially wrap around a body part in a first spiral or circuit, and the second zone 208 refers to the portion of the wrap 210 designed to wrap subsequently around the first spiral (i.e., as an overlapping layer). In other embodiments, there may only be a single tab portion (i.e., second tab portion 230) such that the elongated wrap extends fully to one end of the compression appliance 200 that includes the first zone 206.

FIG. 13 also provides an overview of the relative dimensions of the compressive appliance 200 and relative arrangement of elements included in the compressive appliance 200. For example, the two zones can be understood to be further distinguishable by the spacing between the wire segments in the two zones. In FIG. 13, it can be observed that the first zone 206 generally has a lateral first width W1, and extends longitudinally from the first edge 222 to an optional neck region (“neck portion” 238) where the width of the wrap 210 can optionally narrow to a lateral second width W2. The second zone 208 extends from the neck portion 230 and has an average lateral third width W3. In this case, the first width W1 can be equal to or slightly smaller than the third width W3. In other embodiments, W1 may be significantly smaller than W3, such that at least a peripheral portion of the fabric of second zone will come into direct contact with the user's skin when the compression appliance 200 is fully wrapped. Outside of the neck portion 238, the two wire segments (upper wire segment 202 and lower wire segment 208) can be understood to be in a substantially parallel arrangement across a length of the wrap. Furthermore, second width W2 is smaller than both W1 and W2. In other embodiments, the width of the wrap 210 (e.g., W1, W2, W3) may be substantially uniform or equal across the length of the wrap 210, such that upper edge 282 and lower edge 284 remain substantially parallel.

Each of the two zones is associated with a particular wiring arrangement. In the first zone 206, a first distance D1 between the upper wire segment 202 and the lower wire segment 204 is generally uniform (such that the two wire segments are substantially parallel), until reaching the neck portion 238, when the spacing between the two wire segments begins to widen. For example, as the wrap 210 approaches and extends into second zone 208, the spacing between the two segments has grown to a second distance D2 greater than first distance D1. Thus, the neck portion 238 can also serve as a tangible indicator to a user that upon reaching the neck portion 238, a properly fitted compression appliance should have completed one loop around the user's body part.

Once the spacing grows to a third distance D3—larger than both D1 and D2—in the second zone 208, it may become substantially uniform or equal again. As a general matter, the first zone 206 may be understood to refer to the portion of the wrap 210 in which the spacing between the two wire segments is narrower and the second zone 208 refers to the portion of the wrap in which the spacing between the two wire segments is relatively wider. The actual distance can vary depending on the size of the specific compressive appliance 200, the body part for which it is designed, and the size of the person for whom it is targeted. In other words, if a body part is larger (e.g., a waist), then the first zone and second zone will need to be longer to accommodate the larger circumference being covered, while smaller body parts (e.g., a lower leg) will need to be shorter to accommodate the smaller circumference to be covered. This will also ensure that the first zone and the second zone overlap one another correctly once the compressive appliance is wrapped (i.e., avoiding the first zone overlapping with itself or the second zone overlapping with itself). Furthermore, because a second circuit will always be of a slightly larger circumference (due to the thickness added by the underlying wrap's first circuit corresponding to the first zone), the second zone can also be understood to have a longer length than the first zone.

In addition, in some embodiments, the compressive appliance 200 includes control interface 242 for interacting with the compression appliance 200. For example, control interface 242 can provide basic user controls accessibly embedded in the wrap. A user can interact with the basic user controls via the control interface 242 provided on the wrap and/or a mobile application (e.g., using a phone or other computing device) to instruct the microprocessor to implement a predetermined sequence and pattern of compression based on a selected compression profile and heat setting. The mobile application can be configured to provide substantially similar selectable options as those offered by the control interface 242 and connect to the compression appliance via a communication module of the compression appliance. For example, the electronic assembly can include a communication module that enables a wireless connection using Bluetooth® radio technology, communication protocols described in IEEE 802.11 (including any IEEE 802.11 revisions), Cellular technology (such as GSM, CDMA, UMTS, EV-DO, WiMAX, or LTE), or Zigbee® technology, among other possibilities. In many cases, the communication module is a wireless connection; however, wired connections may also be used. For example, the communication module may include a wired serial bus such as a universal serial bus or a parallel bus, among other connections.

Thus, although not depicted in the drawings, in different embodiments, a user can use the interface provided by a mobile application to change the compression settings. The application (“app”) can offer a user interface that may be accessed via any user computing device configured for connection to a network. In different embodiments, the application can be configured to offer content via native controls presented via an interface. Throughout this application, an “interface” may be understood to refer to a mechanism for communicating content through a client application to an application user. In some examples, interfaces may include pop-up windows that may be presented to a user via native application user interfaces (1.11s), controls, actuatable interfaces, interactive buttons or other objects that may be shown to a user through native application UIs, as well as mechanisms that are native to a particular application for presenting associated content with those native controls. In addition, the terms “actuation” or “actuation event” refers to an event (or specific sequence of events) associated with a particular input or use of an application via an interface, which can trigger a change in the display of the application. This can include selections or other user interactions with the application, such as a selection of an option offered via a native control, or a ‘click’, toggle, voice command, or other input actions (such as a mouse left-button or right-button click, a touchscreen tap, a selection of data, or other input types). Furthermore, a “native control” refers to a mechanism for communicating content through a client application to an application user. For example, native controls may include actuatable or selectable options or “buttons” that may be presented to a user via native application UIs, touch-screen access points, menus items, or other objects that may be shown to a user through native application UIs, segments of a larger interface, as well as mechanisms that are native to a particular application for presenting associated content with those native controls. The term “asset” refers to content that may be presented in association with a native control in a native application. As some non-limiting examples, an asset may include text in an actuatable pop-up window, audio associated with the interactive click of a button or other native application object, video associated with a teaching user interface, or other such information presentation.

In different embodiments, the control interface 242 can be disposed in a predetermined location along or accessible via an outwardly facing surface of the wrap disposed toward second end 254. The control interface 242 is electrically connected to the controller such that the controller can receive input from a user via the control interface 242 and transmit corresponding instructions to the relevant components of the electronic assembly. For example, the control interface 242 can include a power button 244, a function button 246, and a charge indicator 248. The power button 244 is used to turn the compression appliance on and off, while the charge indicator 248 can indicate a remaining charge in the power source. The function button 246 can be configured to select from a plurality of compression profiles and heat settings. In other words, the function button can be configured to instruct the microprocessor to implement a predetermined compression profile and/or heat setting. The function button 246 can be pressed once to select a first predetermined option, long pressed (e.g., longer than three seconds), and/or the function button can be pressed a plurality of times to cycle through additional compression profile and/or heat setting options. In other embodiments, the control interface 242 may include additional or alternative options for interacting with the appliance.

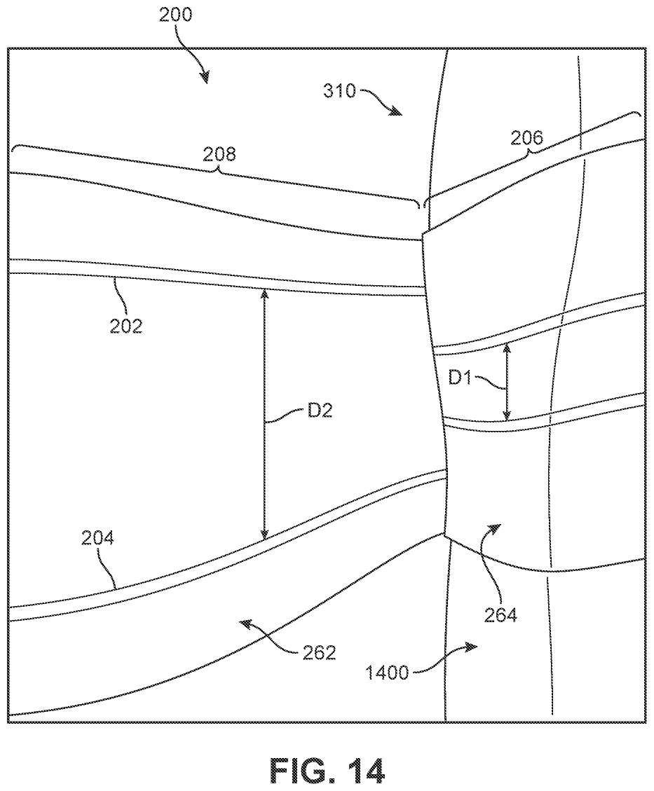

FIG. 14 is a schematic depiction of the compression band of FIG. 13 partially wrapped around the body of a wearer. As shown in FIG. 14, compressive appliance 200 is wrapped around the body 1400 (e.g., leg, arm, waist, etc.) of the wearer. It will be understood that this depiction of partial wrapping is simply to illustrate the effect of the double-wrapping configuration. As shown in FIG. 14, as the second zone 208 is wrapped around the foot, the inner surface 262 of second zone 208 will be placed in contact with the outer surface 264 of the first zone 206. During the subsequent second circuit around the body 1400, the two surfaces will be disposed or pressed against one another. However, due to the variation in spacing in the two zones, the upper wire segment 202 and the lower wire segment 204 in the second zone 208 do not make contact with the previous lengths of the upper wire segment 202 and lower wire segment 204 in the first zone 206. In other words, the wiring extending through second zone 208 remains offset from the wiring extending through first zone 206 when the two zones overlap.

While various embodiments have been described, the description is intended to be exemplary, rather than limiting, and it will be apparent to those of ordinary skill in the art that many more embodiments and implementations are possible that are within the scope of the embodiments. Although many possible combinations of features are shown in the accompanying figures and discussed in this detailed description, many other combinations of the disclosed features are possible. Any feature of any embodiment may be used in combination with or substituted for any other feature or element in any other embodiment unless specifically restricted. Therefore, it will be understood that any of the features shown and/or discussed in the present disclosure may be implemented together in any suitable combination. Accordingly, the embodiments are not to be restricted except in light of the attached claims and their equivalents. Also, various modifications and changes may be made within the scope of the attached claims.

What is claimed is:

1. An article of apparel, comprising:
a garment having a loose-fitting outer shell; and
an integrated compression band within the outer shell and configured to apply compression to a portion of the body of a wearer of the garment to promote muscle recovery;
wherein the compression band is disposed internal to, and separate from, the outer shell of the garment.

2. The article of claim 1, wherein the compression band includes at least one shape memory alloy wire configured to selectively constrict in order to provide the compression to the portion of the body.

3. The article of claim 2, wherein the at least one shape memory alloy wire forms a hoop that is configured to reduce in circumference upon application of an electric current to the shape memory alloy wire.

4. The article of claim 3, further including a control device configured to receive input from a wearer of the article of apparel to activate the at least one shape memory alloy wire.

5. The article of claim 2, wherein the compression band is configured to apply heat to the body of the wearer.

6. The article of claim 1, wherein the compression band is configured to be removed from the garment.

7. The article of claim 6, further including a fastening device disposed within a front pocket of the garment.

8. The article of claim 1, wherein the garment includes at least one tubular sleeve configured to receive a limb of a wearer; and
wherein the compression band is located within the tubular sleeve and is configured to apply compression to the limb of the wearer.

9. An article of apparel, comprising:
a garment having a loose-fitting outer shell; and
an integrated compression band within the outer shell and configured to apply compression to a portion of the body of a wearer of the garment;
wherein the compression band includes at least one shape memory alloy wire configured to selectively constrict in order to provide the compression to the portion of the body; and
wherein the at least one shape memory alloy wire is configured to apply heat to the body of the wearer.

10. The article of claim 9, wherein the at least one shape memory alloy wire forms a hoop that is configured to reduce in circumference upon application of an electric current to the shape memory alloy wire.

11. The article of claim 10, further including a control device configured to receive input from a wearer of the article of apparel to activate the at least one shape memory alloy wire.

12. The article of claim 9, wherein the garment is configured to be worn on the torso of the body.

13. The article of claim 12, further including a fastening device disposed within a front pocket of the garment.

14. The article of claim 9, wherein the garment includes at least one tubular sleeve configured to receive a limb of a wearer; and
wherein the compression band is located within the tubular sleeve and is configured to apply compression to the limb of the wearer.

15. An article of apparel, comprising:
a garment having a loose-fitting outer shell; and
an integrated compression band within the outer shell and configured to apply compression to a portion of the body of a wearer of the garment;
wherein the garment is configured to be worn on the torso of the body; and
further including a fastening device disposed within a front pocket of the garment.

16. The article of claim 15, wherein the compression band includes at least one shape memory alloy wire configured to selectively constrict in order to provide the compression to the portion of the body.

17. The article of claim 16, wherein the at least one shape memory alloy wire forms a hoop that is configured to reduce in circumference upon application of an electric current to the shape memory alloy wire.

18. The article of claim 16, wherein the compression band is configured to apply heat to the body of the wearer.

19. The article of claim 15, wherein the compression band is disposed internal to, and separate from, the outer shell of the garment.

20. The article of claim 19, wherein the compression band is attached to an inner layer of the garment; and
wherein the inner layer of the garment has one or more cutouts exposing the compression band to the body of a wearer of the garment.
